(19) United States
(12) Patent Application Publication
(10) Pub. No.: US 2003/0138949 A1
(43) Pub. Date: Jul. 24, 2003

(54) Methods for the regeneration of pancreatic islets and expansion of pancreatic endocrine cells

(76) Inventors: Anil Bhushan, Los Angeles, CA (US); David Warburton, La Canada, CA (US)

Correspondence Address: MYERS BIGEL SIBLEY & SAJOVEC, PO BOX 37428, RALEIGH, NC, 27627, US

(21) Appl. No.: 10/315,703

(22) Filed: Dec. 10, 2002

Related U.S. Application Data
(60) Provisional application No. 60/341,080, filed on Dec. 12, 2001.

(51) Int. Cl. A61K 38/18; C12N 5/08

(52) U.S. Cl. 435/366; 514/12

(57) ABSTRACT

A method of facilitating the proliferation of pancreatic progenitor cells, particularly mammalian cells, is described. The method is carried out by administering an FGF polypeptide such as FGF-10 polypeptide to the cells in an amount effective to facilitate the proliferation thereof.

### RELATED APPLICATIONS

This application claims the benefit of U.S. provisional application serial. No. 60/341,080, filed Dec. 12, 2001, the disclosure of which is to be incorporated by reference herein in its entirety.

### FIELD OF THE INVENTION

The present invention concerns methods of regenerating pancreatic islet tissue to be used in regeneration or transplant therapies for diabetes mellitus.

### BACKGROUND OF THE INVENTION

The pancreas originates from the dorsal and ventral regions of the foregut endoderm directly posterior to the stomach. The first indication of pancreatic morphogenesis occurs in mice at 15-25 somites or embryonic day (E) 8.5-9.5, when the endoderm evaginates to form buds (Golosow and Grobstein (1962). Dev. Biol. 4, 242-255; Wessels and Cohen (1967). Dev. Biol. 15, 237-270). Subsequently the mesenchyme condenses around the underlying endoderm and the epithelial buds grow in size accompanied by the differentiation of cell types that will constitute the functional units of the pancreas (Pictet and Rutter (1972). In Handbook of Physiology, vol. 1 (ed. Steines and Frenkel), pp. 25-66. Washington: The Williams & Wilkins Co., Baltimore); exocrine cells maintain epithelial characteristics and form branched ducts and acini; endocrine cells detach from the epithelial buds and migrate towards the mesenchyme and cluster together to eventually form islets (Slack (1995). Development 121, 1569-1580). Cell marking and chimeric mouse studies have shown that these differentiated exocrine and endocrine cell types are not determined by lineage, but instead arise from a common population of progenitor endoderm cells (Deltour et al. (1991). Development 112, 1115-1121; Herrera, P. L. (2000). Development 127, 2317-2322; Jensen et al. (2000). Diabetes 49, 163-176; Percival and Slack (1999). Exp. Cell Res. 247, 123-132). Several studies indicate that Pdx1, a homeodomain transcription factor expressed in all epithelial cells in early pancreatic buds, marks this pluripotent cell population (Gradwohl et al. (2000). Proc. Natl. Acad. Sci. USA 97, 1607-1611.; Krapp et al. (1998). Genes Dev. 12, 3752-3763). Consistent with this notion, evagination of the endoderm occurs in Pdx1 null mice, however morphogenesis and differentiation of the pancreatic buds is arrested (Offield et al. (1996). Development 122, 983-995; Jonsson et al. (1994). Nature 371, 606-609). Embryonic tissue recombination studies indicate a cell-autonomous role for Pdx1 in the epithelium to impart competence to respond to mesenchymal signals (Ahlgren et al. (1996). Development 122, 1409-1416).

Inductive signals originating in the mesenchyme have been shown to play an essential role in the development of the pancreatic epithelium (Golosow and Grobstein (1962). Dev. Biol. 4, 242-255; Wessels and Cohen (1967). Dev. Biol. 15, 237-270). These classic studies use recombined embryonic tissues to show that pancreatic buds could develop in vitro, but that isolated epithelium would not undergo any growth or morphogenesis in the absence of mesenchyme. Recent studies indicate that the default state of the epithelium is to form islets, and mesenchyme provides an instructive signal for differentiation of exocrine cells (Gittes et al. (1996). Development 122, 439-447; Miralles et al. (1999). Proc. Natl. Acad. Sci. USA 96, 6267-6272). While these in vitro experiments have been useful in assessing the role of mesenchyme and various growth factors in pancreatic development, they do not allow for the identification of endogenous signaling molecules. In addition, in these experiments the epithelial tissue was isolated after having had extended contact with the mesenchyme, so there was the possibility of an early involvement of mesenchyme in the formation and development of the pancreatic epithelial buds, which was not examined.

### SUMMARY OF THE INVENTION

Genetic methods reveal an essential role for mesenchyme at the earliest stages of pancreatic bud outgrowth. The secreted growth factor, Fgf10 is expressed in the mesenchyme at stages that coincide with the rapid growth of epithelial buds. Using Fgf10−/− embryos, evidence is provided that Fgf10 signalling regulates proliferation of, and, therefore, the size of, the epithelial progenitor cell population marked by PDX1. Loss of this pancreatic progenitor pool led to abnormal differentiation and morphogenesis of the pancreas.

The present invention is based upon the finding that FGF polypeptides are essential for normal islet stem cell maintenance, proliferation and generation of islets. Hence, a first aspect of the invention concerns the use of FGF signaling pathway elements such as ligands, receptors and intracellular components to activate pancreatic stem cells for the purpose of islet regeneration or transplantation therapy for diabetes mellitus.

A further aspect of the present invention is a method of facilitating the proliferation of pancreatic cells, the method comprising administering an FGF polypeptide to said cells in an amount sufficient to facilitate the proliferation thereof.

### BRIEF DESCRIPTION OF THE DRAWINGS

The patent or patent application file contains at least one drawing executed in color. Copies of the patent or patent application publication with color drawing(s) will be provided by the U.S. Patent and Trademark Office upon request and payment of the necessary fee.

FIG. 1. Fgf10 is expressed in the pancreatic mesenchyme during the early stages of pancreatic organogenesis. (A,B,E) Whole-mount in situ hybridization on dissected gastrointestinal tracts (dorsal is to the right) showing (A) Fgf10 expression in the gut dissected from an E9.5 embryo. The first signs of Fgf10 expression in the posterior foregut were two distinct stripes where the dorsal and ventral pancreatic buds emerge. Expression of Fgf10 in the foregut anterior to the pancreas was also observed in the lung buds. (B) By E10.5, the dorsal epithelial bud was clearly visible and Fgf10 was expressed broadly in the surrounding mesenchyme. Expression of Fgf10 also extended into the posterior stomach mesenchyme. (C,D) Vibratome sections (20 μm) of the gut from the E10.5 embryo shown in B demonstrates that Fgf10 expression is confined to the mesenchyme adjacent to the dorsal (C) and ventral (D) bud. (E) Fgf10 continued to be expressed at E11.5 in a restricted area in the dorsal mesenchyme (indicated by an arrow). The uniform weak staining observed in the stomach epithelium was due to the trapping of the in situ probe in the lumen of the stomach. d, dorsal; v, ventral; db, dorsal bud; vb, ventral bud; lb, lung bud; st, stomach.

FIG. 2. Pancreatic hypoplasia and absence of islet cells in Fgf10−/− embryos. (A) Gastrointestinal tract from an E17.5 wild-type embryo. (B) Gross appearance of pancreatic region dissected from an E17.5 wild-type embryo and (C) a schematic representation illustrating the pancreas. The pancreatic tissue at this stage is localised near the spleen, which derived from the dorsal bud (green), and along the duodenum, which derived from the ventral bud (blue). (D) Haematoxylin and Eosin staining of the pancreas tissue from an E17.5 wild-type embryo showing the presence of acini exocrine tissue and heavily nucleated clusters of islet cells. (E) The islet clusters express insulin (green) and glucagon (red). (F) Exocrine tissue expresses carboxypeptidaseA (green). (G) The gastrointestinal tract from E17.5 Fgf10−/− embryos were overtly similar to that of wild-type littermates except for a smaller stomach. (H) The pancreatic tissue in the mutant Fgf10 embryos was drastically reduced (I) although present in both the splenic (green) and duodenal (blue) locations. (J) Haematoxylin and Eosin staining shows the presence of acinar tissue but no islet clusters are evident. (K) Scattered insulin (green) and glucagon-expressing (red) cells are present. Co-expression of insulin and glucagon indicates that these endocrine cells are immature (arrow). (L) The acini from the mutant Fgf10 embryo stained for the exocrine marker, carboxypeptidaseA. CA, carboxypeptidaseA; ins, insulin; glu, glucagon; st, stomach; sp, spleen.

FIG. 3. The size of the pancreatic epithelium in Fgf10−/− embryos is greatly reduced. (A) Hnf3β whole-mount in situ hybridization to the epithelium of the foregut of wild-type E11.5 embryos. (B) Hnf3β expression in the pancreatic region and posterior stomach was greatly reduced in the foregut of Fgf10 mutant embryos. (C) ISL1 was expressed predominantly in the dorsal mesenchyme and a few differentiating endocrine cells in the pancreatic epithelium from E10.5 wild-type embryos. The dorsal bud is oriented to the top. (D) Strong expression of ISL1 was observed in the dorsal mesenchyme of Fgf10−/− embryos at E10.5, although very few scattered cells within the epithelium that expressed ISL1 were detected. (E) Sagittal sections of the dissected gut from wild-type embryo stained with carboxypeptidaseA (green) and glucagon (red). The branched morphology of both dorsal and ventral pancreatic buds is evident. (F) Sagittal section of dissected gut from an Fgf10−/− embryo shows that the formation of both dorsal and ventral buds occurred, however, no branching of the epithelium is apparent. (G) Transverse sections of the dorsal bud of E13.5 wild-type embryo stained for the pan-epithelial markers cytokeratin (red) and glucagon (green). The dorsal pancreatic bud of a wild-type embryo shows a characteristic highly branched epithelium. (H) The dorsal bud of the Fgf10−/− embryo has a small pancreatic epithelium and no branching of the epithelium is visible. Differentiation of early endocrine cells, as identified by glucagon staining, occurred in the mutant embryos and these cells are seen typically clustered together. db, dorsal pancreatic bud; vb, ventral pancreatic bud; ISL1, Islet1; CA, carboxypeptidaseA; du, duodenum.

FIG. 4. Fgf10−/− embryos have a small pancreatic primordium because the Pdx1 -expressing epithelial progenitor cell population is not maintained. Dorsal is to the right. (A) Pdx1 is expressed uniformly in undifferentiated cells throughout the developing pancreatic buds in wild-type embryos at E10.5 (B) Immunofluorescence analysis of PDX1 expression in transverse section of an E10.5 wild-type embryo shows the buds emerging from the foregut. (C) By 12.5, Pdx1 expression is no longer uniform (due to differentiation of precursor cells) and the branching of the epithelium is evident in the dorsal bud. (D) By E13.5, Pdx1 expression is increasingly restricted within the epithelium and accentuates the lobulated structure of both the pancreatic buds. As compared to the wild-type littermates the Pdx1 expression in the Fgf10−/− embryos at E10.5 (E) identifies the formation of two small but distinct pancreatic buds. (F) Immunofluorescence analysis confirmed the reduced expression of PDX1 in these two pancreatic buds in mutant embryos. Pdx1 expression is no longer observed later in development at E12.5 (C) and E13.5 (H). Arrows indicate the area within the gut where the pancreatic buds normally form. Occasionally, some weak expression of Pdx1 was observed in the ventral bud of mutant embryos (H). v, ventral; d, dorsal.

FIG. 5. The smaller pancreatic epithelium in the Fgf10 mutants was primarily due to the decreased proliferation of progenitor cells that are marked by expression of PDX1. (A,B) Immunofluorescence analysis for the expression of PDX1 (green) and glucagon (red) in the pancreatic epithelium of E11.5; (A) wild-type littermate and (B) Fgf10−/− embryo. The pancreatic epithelium in the Fgf10 mutant embryo is reduced in size, although no concomitant increase in glucagon expression is evident. In addition, no glucagon-positive cells are evident in the ventral bud of the Fgf10−/− embryo. (C-H) Analysis of proliferating precursor epithelial cells in the dorsal bud of Fgf10−/− (D,F,H) and wild-type (C,E,G) littermate embryos at E10.5. PDX1 labelling identifies the dorsal pancreatic epithelium, which is smaller in Fgf10−/− (D) compared to wild-type littermates (C). (F) BrdU labelling shows very few proliferating cells within the dorsal pancreatic epithelium of Fgf10−/− embryos. (E) In wild type a large fraction of dorsal pancreatic epithelial cells have gone through S-phase and stained BrdU positive. The outline designates the boundary of the dorsal epithelial bud. (G,H) Merged images show double labelling for PDX1 and BrdU and confirm that the proliferating epithelial cells also express PDX1 (magnified 2×). To quantify the difference in proliferation of precursor epithelial cells between Fgf10−/− embryos and their wild-type littermates, four consecutive sections from each of four wild-type and four Fgf10 mutants were used to calculate the proliferative index (BrdU+/PDX1+)×100. Using such analysis, wild-type embryos displayed an average proliferative index of 50.4±3.8 (n=4) as compared with 15.1±2.9 (n=4) for Fgf10−/− embryos.

FIG. 6. Exogenously added FGF10 can rescue the PDX1-expressing pool of epithelial cells in cultured pancreas derived from Fgf10 mutants. Consecutive sections of explanted cultured tissue were stained for PDX1 (green) and glucagon (red). (A) Cultured explanted tissue derived from an Fgf10 mutant stained for PDX1. Very few stained cells were detected. (B) Section adjacent to A showing that glucagon-positive cells were present. (C) Explanted tissue derived from Fgf10 mutants, cultured with 50 ng/ml FGF10 and stained for PDX1. The PDX1-positive cells are present in the bud outgrowth from the foregut epithelium. (D) Glucagon-expressing cells in an adjacent section to C. (E) An additional population of PDX1-positive cells detected in the explanted tissue from Fgf10 mutants that was cultured with FGF10. (F) No glucagon-positive cells were detected in the section adjacent to that in E. (G) Cultured explanted tissue derived from a wild-type littermate and stained for PDX1. The arrow indicates an additional population of PDX1-stained cells. (H) Glucagon-positive cells in the section adjacent to that in G. The white dashed lines in D, F and H indicate the position of the PDX1-positive cells in C, E and G, respectively.

### DESCRIPTION OF THE PREFERRED EMBODIMENTS

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Applicants specifically intend that the disclosure of all United States patent references cited herein be incorporated herein by reference in their entirety.

Cells useful for carrying out the methods of the present invention may be of any species of origin, but are preferably mammalian cells, including but not limited to dog, cat, horse, pig, goat, monkey or human cells. In one embodiment, human cells are preferred.

Cells used to carry out the present invention are, in general, pancreatic cells, and are in general pancreatic progenitor or stem cells. Such cells may be obtained and produced by any suitable procedure, including the procedures described herein and procedures known in the art. In general, the stem cells of the invention are not embryonic stem cells, but are rather progenitor cells that give rise to a particular type or category of progeny cells (e.g. pancreatic stem cells used in the invention could give rise to acinar cells, islet cells, and/or ductal cells, etc.). Examples of suitable stem cells for carrying out the present invention, and/or manners of isolating the same, include but are not limited to those described in: Ramiya, V. K., et al., Reversal of insulin-dependent diabetes using islets generated in vitro from pancreatic stem cells. Nature Medicine 6 278-282 (2000); U.S. Pat. No. 6,303,355 to Opara., (Method for culturing, cryopreserving, and encapsulating pancreatic islet cells); U.S. Pat. No. 6,129,911 to Faris, “Liver Stem Cell”; U.S. Pat. No. 6,023,009 to Stegemann et al., “Artificial Pancreas”); U.S. Pat. No. 6,001,647 to Peck et al. (In vitro growth of functional islets of Langerhans and in vivo uses thereof); U.S. Pat. No. 5,919,703 to Mullen et al., (Preparation and storage of pancreatic islets); U.S. Pat. No. 5,888,705, Rubin et al.; (Compositions and method of stimulating the proliferation and differentiation of human fetal and adult pancreatic cells ex vivo); U.S. Pat. No. 5,855,616 to Fournier et al. (Bioartificial pancreas); U.S. Pat. No. 5,681,587, Halberstadt et al. (Growth of adult pancreatic islet cells); U.S. Pat. No. 5,587,309, Rubin et al. (Method of stimulating proliferation and differentiation of human fetal pancreatic cells ex vivo).

Fibroblast growth factors (FGFs) are a family of proteins characteristic of binding to heparin and are, therefore, also called heparin binding growth factors (HBGF). Expression of different members of these proteins are found in various tissues and are under particular temporal and spatial control. These proteins are potent mitogens for a variety of cells of mesodermal, ectodermal, and endodermal origin, including fibroblasts, corneal and vascular endothelial cells, granulocytes, adrenal cortical cells, chondrocytes, myoblasts, vascular smooth muscle cells, lens epithelial cells, melanocytes, keratinocytes, oligodendrocytes, astrocytes, osteoblasts, and hematopoietic cells. Each member has functions overlapping with others and also has its unique spectrum of functions. In addition to the ability to stimulate proliferation of vascular endothelial cells, both FGF-1 and 2 are chemotactic for endothelial cells and FGF-2 has been shown to enable endothelial cells to penetrate the basement membrane. Consistent with these properties, both FGF-1 and 2 have the capacity to stimulate angiogenesis. Another important feature of these growth factors is their ability to promote wound healing. Many other members of the FGF family share similar activities with FGF-1 and 2 such as promoting angiogenesis and wound healing. Several members of the FGF family have been shown to induce mesoderm formation and to modulate differentiation of neuronal cells, adipocytes and skeletal muscle cells. Other than these biological activities in normal tissues, FGF proteins have been implicated in promoting tumorigenesis in carcinomas and sarcomas by promoting tumor vascularization and as transforming proteins when their expression is deregulated.

The FGF family includes, among others, basic FGF, acidic FGF, int 2, hst 1/k-FGF, FGF-3, FGF-5, FGF-6, FGF-7, keratinocyte growth factor, AIGF (FGF-8), FGF-10, FGF-11, FGF-13, and FGF-15. Two of the members, FGF-1 and FGF-2, have been characterized under many names, but most often as acidic and basic fibroblast growth factor, respectively. The normal gene products influence the general proliferation capacity of the majority of mesoderm and neuroectoderm-derived cells. They are capable of inducing angiogenesis in vivo and may play important roles in early development (Burgess, W. H. and Maciag, T., Annu. Rev. Biochem., 58:575-606, (1989)). Numerous examples of FGFs are known and described in, among other locations, U.S. Pat. No. 5,891,848, U.S. Pat. No. 5,859,208, U.S. Pat. No. 6,274,712, U.S. Pat. No. 6,110,893, U.S. Pat. No. 6,013,477, U.S. Pat. No. 5,962,323, U.S. Pat. No. 5,750,365, U.S. Pat. No. 5,728,546, and U.S. Pat. No. 5,714,458. (applicants specifically intend that the disclosures of all United States patent references cited herein be incorporated by reference herein in their entirety). FGF-2, FGF-7 and FGF-10 are currently preferred.

Fibroblast growth factor 10 (FGF-10) used to carry out the present invention is known and described in, for example, U.S. Pat. No. 5,817,485 to Hu et al., PCT Application WO97/20929 to Itoh et al., and UK Patent Application GB 2321852 to Hamada and Makino. The FGF-10 may be of any species of origin, but is preferably mammalian and in one embodiment is human. The FGF-10 may be naturally occurring or produced by recombinant means. The FGF-10 may be in the native form or contain various modifications, such as the substitution or deletion of amino acids therein, or the addition of solubilizing groups such as polyethylene glycol, (PEG) so long as the FGF-10 contains sufficient activity as found in the parent molecule to achieve the effect of facilitating the proliferation of cells as described herein.

As used herein, an “FGF polypeptide” (whether referring to FGFs in general or a particular FGF in particular such as an “FGF-10 polypeptide) is any polypeptide that has an amino acid sequence that is the same as, or substantially identical to, all or a portion of a/the naturally occurring FGF protein and which has substantially the same function as a/the natural or full-length recombinant FGF as described herein with respect to inducing the proliferation of pancreatic progenitor cells. Thus, the term includes naturally occurring FGF, recombinant FGF, FGF analogs (e.g., mutant forms of FGF, and natural or synthetic polypeptide fragments of the full-length FGF protein and analogs, as long as these analogs and fragments have substantially the same function as natural or full-length recombinant aFGF with respect to pancreatic progenitor cells as described herein. These analogues and fragments can easily be tested for their function by using the culture methods described herein.

FGF polypeptides may be administered to cells in vitro by simply adding the FGF polypeptide to the growth or culture media in which such cells are being sustained. The collection, culturing and growth of pancreatic cells in such media is known, and described in, for example, U.S. Pat. No. 6,303,355 to Opara; U.S. Pat. No. 5,387,237 to Fournier et al., U.S. Pat. 5,614,205 to Usala, and those other references cited above. The FGF polypeptide may be included in the growth media or culture media in any amount effective to facilitate the growth or proliferation of the cells, and in general may be included in an amount ranging from about 0.5, 1, 5, or 10 ng/ml up to about 200, 300, 500 or 1000 ng/ml, or more. An amount of 50 ng/ml is currently preferred.

Cells produced by the methods of the present invention may be further processed, encapsulated if desired and implanted into any suitable subject for human or veterinary therapeutic treatment, such as for the treatment of type II diabetes. Suitable subjects including but not limited to dog, cat, horse, goat, pig, monkey and human subjects. In one embodiment, the cells implanted are of the same species as the subject into which the cells are implanted.

The present invention is explained in greater detail in the Examples set forth below.

### EXAMPLES

1. Materials and Methods

Generation and Genotyping of Fgf10 Mutant Mice.

Targeted disruption of Fgf10, and genotyping of offspring by PCR analysis of genomic DNA have been described previously (Sckinc et al. (1999). Nat. Genet. 21, 138-141). All mice were bred on a C57BL/6 genetic background.

RNA In Situ and Immunohistological Analysis.

Gastrointestinal tract that included the lung, stomach, pancreas and duodenum were dissected and fixed by immersion in either 4% neutral buffered paraformaldehyde (for RNA in situ analysis and TUNEL) or in Bouin's fixative (for immunohistochemistry and bromodeoxyuridine (BrdU) detection). Whole-mount in situ hybridization was performed using standard protocols. The Pdx1 probe was provided by Dr C. Wright. Unless otherwise noted, gastrointestinal tracts were oriented so that sections were cut along the anterior to posterior axis. For general histology, sections were stained with Haematoxylin and Eosin. Immunofluorescence analysis was performed on 6 μm paraffin sections essentially as described previously (Miralles et al. (1999). Proc. Natl. Acad. Sci. USA 96, 6267-6272). The primary antibodies used in this assay were the following: guinea pig anti-insulin, diluted 1:200 (DAKO); mouse anti-glucagon, diluted 1:2000 (Sigma); rabbit anti-carboxypeptidaseA, diluted 1:200 (Biogenesis); mouse anti-pan-cytokeratin derived from the PCK-26 hybridoma, diluted 1:50 (Sigma); rabbit anti-PDX1, diluted 1:500 (gift from J. Habener); mouse anti-ISL1 (39.4D5) diluted 1:10 (Developmental Hybridoma Bank). The secondary antibodies used were diluted as follows: FITC-conjugated anti-rabbit, anti-guinea pig, diluted 1:200 (Jackson Laboratory); Rhodamine-conjugated anti-mouse, anti-rabbit, diluted 1:200 (Jackson Laboratory). TUNEL assay on paraffin sections was performed using a commercially available kit (Roche).

BrdU Detection and Cell Counting.

BrdU labelling was initiated by intraperitoneal injection (50 μg/g body weight) 30 minutes before sacrifice of the pregnant mother. Embryos were dissected and processed as described above. Double immunofluorescence analysis for PDX1/BrdU was performed; BrdU was revealed using anti-BrdU (Amersham). The number of PDX1-positive cells and PDX1-/BrdU-positive cells in the dorsal pancreatic buds were counted and the percentage of BrdU incorporated calculated (proliferative index). For cell quantification, four consecutive sections from each of four wild type and four Fgf10 mutants were analyzed in this manner, giving a total of 32 data points. Statistical significance was determined using Student's t-test.

In Vitro Organ Cultures.

Gastrointestinal tract was dissected from E9.5 embryos and cultured in three-dimensional collagen gels with RPMI medium containing 1% calf serum. In some cases the medium was supplemented with 50 ng/ml human recombinant FGF10 (R&D) every 24 hours. Cultures were maintained at 37° C. in a humidified 5% CO2 incubator. After 48 hours, the tissue was fixed in Bouin's fixative, embedded in paraffin wax and processed for immunohistology as described above.

2. Results

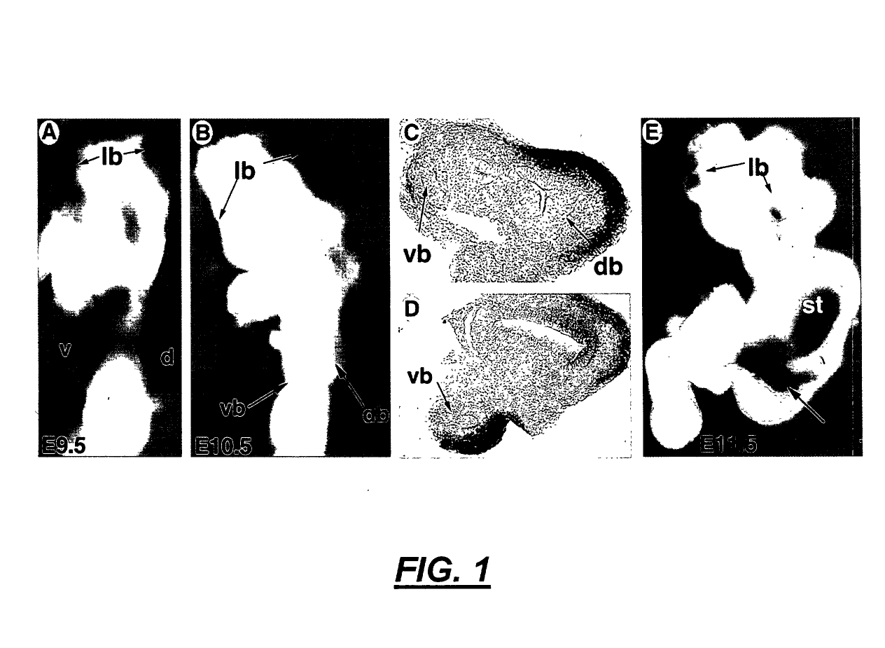

In order to identify secreted growth factors expressed in the mesenchyme during the earliest stages of pancreatic development, E10.5 pancreatic mesenchyme was analyzed by RT-PCR. Fgf10 was expressed at high levels, leading us to characterize its expression in detail by in situ hybridisation. Fgf10 is expressed at E9.5 in two thin stripes in the posterior foregut, the area of the gut tube where the pancreatic buds form (FIG. 1A). Within the anterior foregut, expression is also observed where the lung buds form (Bellusci et al. (1997). Development 124, 4867-4878). In both regions, Fgf10 expression was restricted to the mesenchyme that had condensed around the developing gut tube. At E10.5, Fgf10 expression had expanded to a broad area within the mesenchyme surrounding the developing pancreatic buds and caudal stomach but not in the rostral duodenum (FIG. 1B). Examination of sections of E10.5 gut tube confirmed that the expression of Fgf10 was restricted to the mesenchyme and surrounded both pancreatic epithelial buds (FIGS. 1C,D). By E11.5, Fgf10 was still expressed at high levels in the mesenchyme located directly above the growing dorsal epithelial bud, but only low levels were detected around the ventral bud (FIG. 1E). By E12.5, virtually no Fgf10 expression was detected around either bud (not shown). Fgf10 expression was not detected in the pancreas during later stages of embryogenesis, although FGF10 expression has been reported in the adult islet cells (Hart et al. (2000). Nature 408, 864-868). Thus during embryogenesis, Fgf10 is transiently expressed in pancreas-associated mesenchyme during early stages of epithelial bud formation.

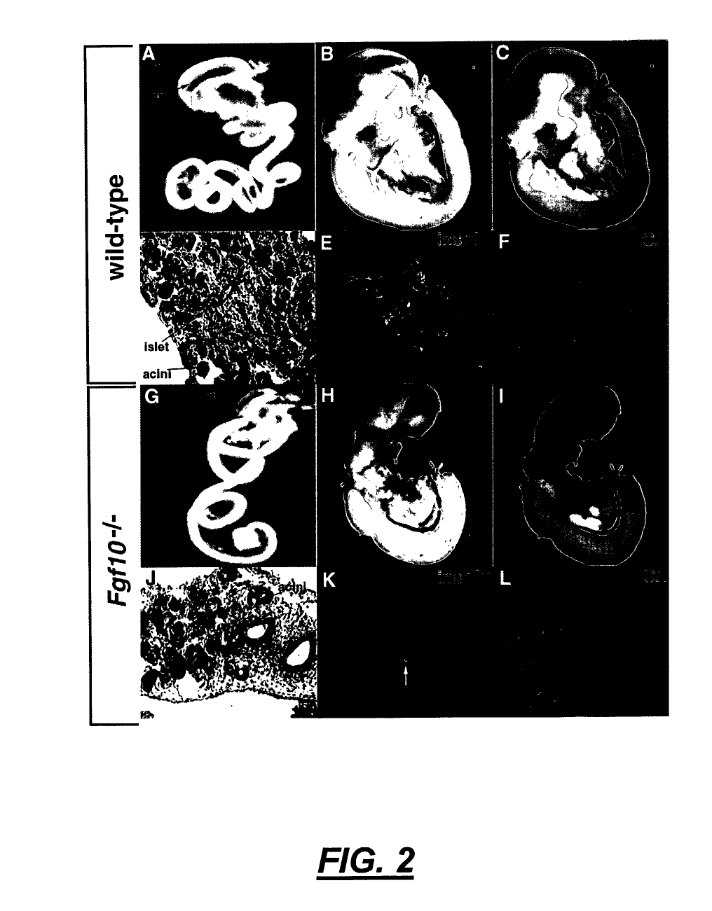

The spatially and temporally specific expression of Fgf10 in the mesenchyme surrounding the growing epithelial buds led us to examine a role for Fgf10 in the development of the pancreas by analyzing mice in which Fgf10 had been inactivated by homologous recombination in ES cells (Min et al. (1998). Genes Dev. 12, 3156-3161; Sekine et al. (1999). Nat. Genet. 21, 138-141). In E17.5 wild-type embryos, the pancreas consisted of prominent glandular tissue near the spleen that derives from the dorsal bud. The ventral bud contributed to the diffuse multilobulated tissue along the duodenal region (Jensen et al. (2000). Nat. Genet. 24, 36-44) (FIGS. 2B,C). Histological analysis of the pancreatic tissue at this stage allowed identification of exocrine acini cells arranged in grape-like clusters as well as some heavily nucleated spheroidal clusters of endocrine islets (FIG. 2D). These islet cells can be more clearly distinguished by immunostaining for endocrine hormones: a classic pattern of insulin-expressing cells at the center of the islet cluster surrounded by glucagon-expressing cells on the periphery of the islet structure (FIG. 2E). The acinar cells, in contrast, expressed carboxypeptidaseA, a marker of differentiated exocrine cells (FIG. 2F). E17.5 Fgf10 mutant littermates had an apparently normal gastrointestinal tract except for the stomach and pancreatic areas, which were reduced in size (FIGS. 2A,G). The spleen, an organ that derives from dorsal mesenchyme was present in mutant embryos (FIG. 2G). Analysis of the pancreas in the Fgf10 mutant embryos revealed rudimentary glandular tissue in both splenic and duodenal regions, indicateing that the pancreatic tissue had derived from both buds (FIGS. 2H,I). The rudimentary pancreatic tissue was devoid of any spheroidal islet clusters, although some well-formed acinar tissue containing zymogen granules was clearly evident (FIG. 2J). Immunohistological analysis of the pancreatic tissue revealed a few scattered glucagon and insulin-positive cells confirming the lack of islet formation in the Fgf10 mutant pancreas. The insulin-positive cells often co-stained for glucagon (FIG. 2K) indicateing that the few insulin cells that do differentiate are abnormal. The acinar tissue by contrast, stained for carboxypeptidaseA, indicating that exocrine cell differentiation and morphogenesis occur in the mutant pancreas, although in drastically reduced amount compared to the wild-type littermate (FIG. 2I).

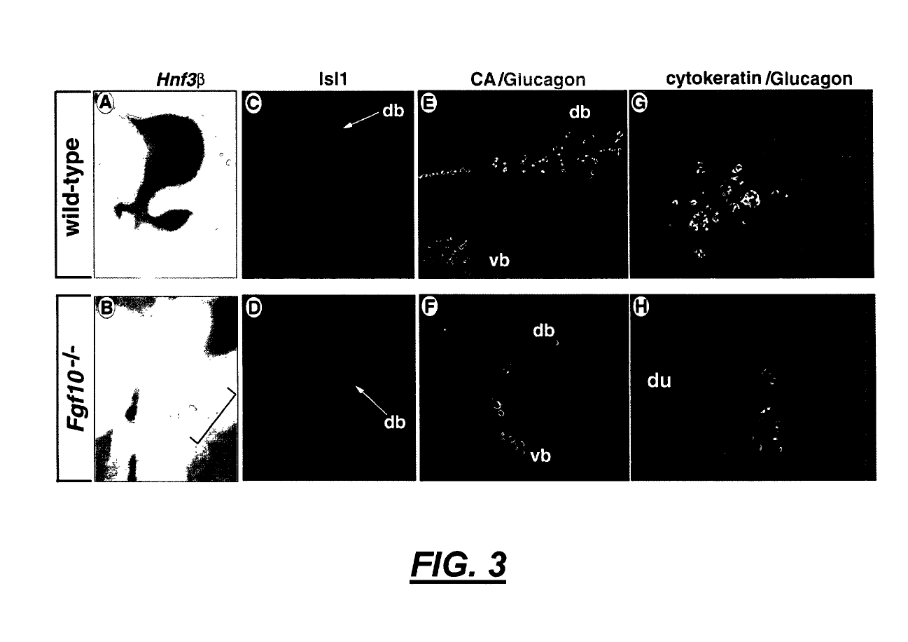

The reduced size of the posterior foregut area where the pancreas forms indicates that early deficiencies in the formation of the foregut may account for the observed phenotype. To investigate the early stages in foregut epithelial development, we the expression of Hnf3β, a member of the winged helix family of transcription factors, was examined. Hnf3β is expressed in the developing gut tube endoderm and serves as a marker for all budding epithelial tissue (Ahlgren et al. (1996). Development 122, 1409-1416). Whole-mount in situ hybridization on E11.5 gastrointestinal tracts confirmed that Hnf3β is expressed in the epithelial tissue of the stomach, pancreatic buds and the duodenum (FIG. 3A). In Fgf10−/− embryos, the epithelial tissue around a limited area of the posterior foregut, including the caudal portion of the stomach and the pancreatic buds, was greatly reduced; the epithelial tissue from other parts of the gut, such as anterior stomach and duodenum, appeared to form normally (FIG. 3B, shown partially). This area of reduced epithelial tissue in the mutant embryos corresponded to the domain of Fgf10 expression in the surrounding mesenchyme observed in wild-type embryos. To examine the state of the mesenchyme surrounding the posterior foregut region, the expression of ISL1, a homeodomain protein and a dorsal mesenchyme marker, was analyzed at E10.5. At this stage, ISL1 is expressed throughout the dorsal mesenchyme of the posterior foregut and in a few endocrine cells within the pancreatic epithelium (Ahlgren et al. (1997). Independent requirement for ISL1 in formation of pancreatic mesenchyme and islet cells. Nature 385, 257-260) (FIG. 3C). The general architecture of tile mesenchyme and the expression of ISL1 were unaffected in Fgf10−/− embryos (FIG. 3D). These results indicate that FGF10 signalling, which originates in the mesenchyme, targets the adjacent epithelium. This hypothesis is consistent with the epithelial localization of FGFR2b, a receptor that can mediate FGF10 signalling during early pancreatic development (Finch et al. (1995). Dev. Dyn. 203, 223-240; Miralles et al. (1999). Proc. Natl. Acad. Sci. USA 96, 6267-6272; Orr-Urtreger et al. (1993). Dev. Biol. 158, 475-486).

Next, the morphology and the differentiation that occurred in the limited pancreatic epithelium that formed in Fgf10−/− embryos was explored. By E13.5, in wild-type embryos, the epithelium undergoes morphogenetic movements referred to as branching morphogenesis that leads to the formation of a ductal network in the pancreas. Differentiation of exocrine cells and some endocrine cells is apparent at this stage although most mature endocrine cells appear after E14.5. The expression of carboxypeptidaseA was utilized to identify differentiated exocrine cells and as a marker for the branching pattern of the epithelium within the pancreatic bud. Sagittal sections across both buds of wild-type littermates displayed the typical branching of the pancreatic epithelium (FIG. 3E). The staining pattern indicates that the epithelium initially elongates before undergoing repeated branching to form a larger network of branched epithelium. Similar sections from Fgf10 null embryos showed that both dorsal and ventral buds had formed; the elongation of the epithelium was initiated, however this epithelium lacked branched structures (FIG. 3F). To further investigate the morphology of the pancreatic epithelium, a pan-epithelial marker, cytokeratin was utilized. Transverse sections of dissected E13.5 gastrointestinal tracts were analyzed for cytokeratin expression by immunofluorescence and co-stained with glucagon to localize the pancreatic epithelium (FIGS. 3G,H). Dorsal buds of wild-type embryos had dispersed glucagon cells that had migrated away from the highly branched epithelium. In contrast, very little epithelium formed in the dorsal bud of Fgf10 mutant embryos and this formed a single tube lacking any branched structure (FIG. 3H). Differentiated glucagon-positive endocrine cells were evident in the mutant pancreas, however, unlike in the wild type, these cells were typically clustered together. Thus, in the Fgf10−/− embryos, formation of dorsal and ventral buds are initiated, as is the differentiation of both endocrine and exocrine cells. However, the pancreatic epithelium is significantly reduced in size and fails to undergo branching.

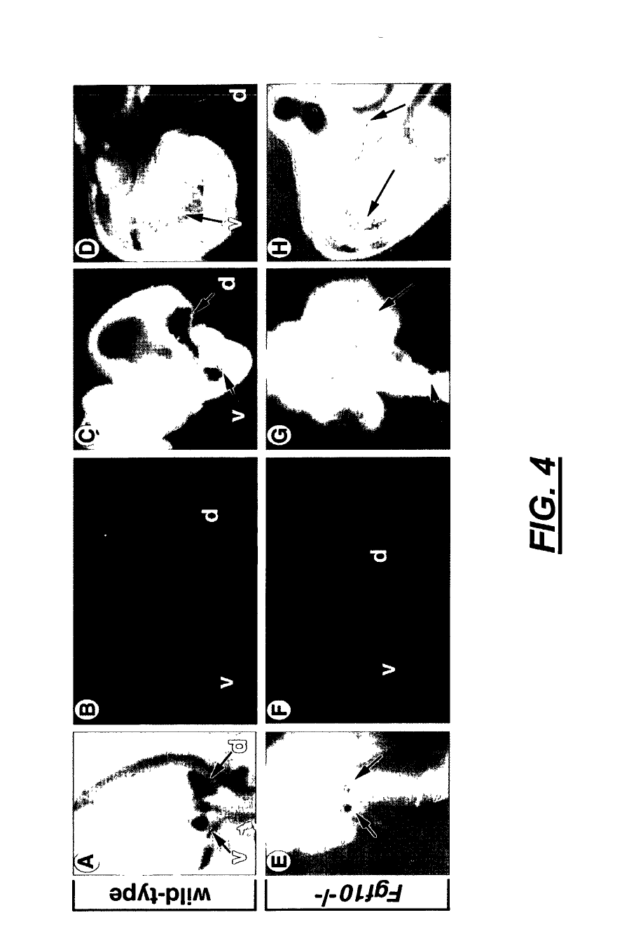

The severe reduction of the pancreatic epithelium in the Fgf10−/− embryos indicates a defect in the formation of the epithelial progenitor cells. Epithelial progenitor cells can be distinguished by the expression of Pdx1, which is initiated when the foregut endoderm is committed to a pancreatic fate. Pdx1 continues to be expressed uniformly in all epithelial cells of the early developing pancreatic buds (Ahlgren et al. (1996). Development 122, 1409-1416; Guz et al. (1995). Development 121, 11-18; Jonsson et al. (1994). Nature 371, 606-609). At E10.5, Pdx1 was expressed throughout the pancreatic epithelium in both buds: the dorsal bud was elongated and larger than ventral bud (FIGS. 4A,B). At later stage (E12.5), Pdx1 continued to be expressed in the pancreas when branching of the dorsal bud epithelium is discernible (FIG. 4C). Weaker Pdx1 expression was also observed in the regions flanking the pancreatic buds, namely the caudal portions of the stomach and the rostral portion of the duodenum. By E13.5, Pdx1 expression highlighted the lobulated structures of both dorsal and ventral pancreatic buds (FIG. 4D). In Fgf10−/− embryos, Pdx1 expression at E10.5 was significantly reduced, nevertheless, distinct dorsal and ventral sites of expression were clearly observable (FIG. 4E). Transverse sections of E10.5 embryos analyzed for PDX1 expression confirmed the formation of two buds that contained far fewer PDX1-positive cells compared to the wild-type littermates' pancreas (FIG. 4F). At later stages in Fgf10−/− embryos, Pdx1 expression was no longer observed in the dorsal pancreatic region, although very weak expression of Pdx1 was observable in the ventral pancreas (FIGS. 4G,H). Thus the specification of PDX1-positive cells occurred in the absence of Fgf10. However, the maintenance of PDX1-positive cells was clearly dependent on FGF10 signalling from the mesenchyme. These results imply a requirement for FGF10 signalling in maintaining/expanding the progenitor cell population marked by PDX1 during the early stages of pancreatic organogenesis.

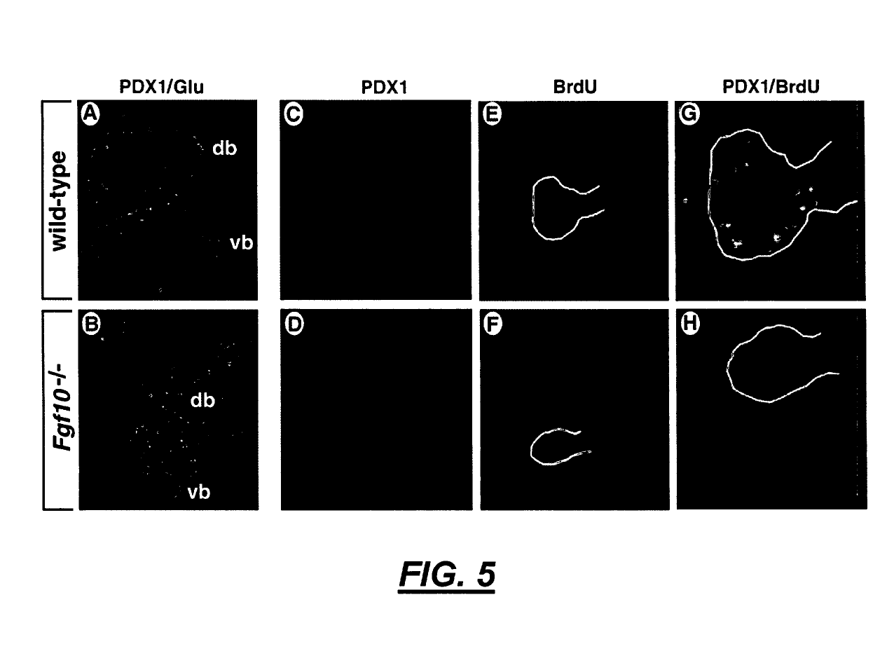

A number of possibilities by which FGF10 could maintain/expand the pancreatic epithelial progenitor population were investigated. FGF10 could act as a factor required to keep the epithelial progenitors in an undifferentiated state, as a survival factor required to inhibit apoptosis or as a mitogenic factor required to simulate proliferation. To investigate these possibilities, it was first assessed whether FGF10 keeps the epithelial progenitors in an undifferentiated state. If this were the case, absence of FGF10 would result in premature differentiation of the precursor epithelial cells that would deplete the progenitor pool of cells. The earliest differentiated cells to emerge during normal pancreatic development are glucagon-expressing cell (Pictet et al. (1972). Dev. Biol. 29, 436-467) and increased early differentiation of precursor epithelial cells would result in an excessive number of glucagon-positive cells at the expense of PDX1-expressing cells (Apelqvist et al. (1999). Nature 400, 877-881; Jensen et al. (2000). Nat. Genet. 24, 36-44). While glucagon-positive cells during normal pancreatic development are readily discernible in the dorsal bud as early as E9, ventral bud expression begins only around E11. To look for premature differentiation in both pancreatic buds, E11.5 embryos were analyzed for the expression of glucagon and PDX1 expression (FIG. 5A). As already shown (FIG. 4), few PDX1-positive cells were observed in the dorsal buds of the Fgf10−/− embryos, however, no concomitant increase in glucagon-positive cells was evident (FIG. 5B). Significantly, no precocious glucagon-positive cells were seen in the ventral bud of the Fgf10−/− embryo (FIG. 5B). Furthermore, no excessive expression of other early markers for endocrine cells, ISL1 and neurogenin 3 (Apelqvist et al. (1999). Nature 400, 877-881; Jensen et al. (2000). Diabetes 49, 163-176; Schwitzgebel et al. (2000). Development 127, 3533-3542) were observed within the Fgf10 mutant pancreatic epithelium consistent with absence of premature endocrine cell differentiation (FIG. 3D and data not shown). These observations indicate that premature differentiation of endocrine cells is unlikely to be the reason for the decreased number of PDX1-positive cells. These results argue against a role for FGF10 in inhibiting differentiation within the pancreas. It was next asked whether FGF10 is required for pancreatic epithelial cell survival. Apoptosis could lead to depletion of cells, however, no increase in apoptosis was observed within the E10.5 Fgf10−/− pancreatic epithelium as determined by Td1-mediated dUTP nick end labelling (TUNEL) assay, indicating that progenitor cells, at least at this stage, were not lost by apoptosis.

It was next examined whether FGF10 is required for the proliferation of the epithelial progenitor cells by determining the extent of cell proliferation within the population of PDX1-positive cells. To assess proliferation levels, DNA synthesis was assayed in the pancreatic epithelium of E10.5 embryos by BrdU incorporation. In pancreatic epithelium from wild-type embryos, a high percentage of PDX1-labelled cells along the rim of the growing bud adjacent to the mesenchyme had gone through S-phase and were, therefore, BrdU positive (FIGS. 5C,E,G). In contrast, very few PDX1-positive cells had incorporated BrdU in the pancreatic epithelium from Fgf10−/− mutant embryos (FIGS. 5D,F,H). This defect in proliferation was restricted to the epithelium since BrdU incorporation in the mesenchyme of Fgf10−/− embryos did not appear to be affected (FIGS. 5E,F). To quantify the proportion of proliferating pancreatic epithelial cells, the number of nuclei, out of all PDX1-positive cells, that had incorporated BrdU were calculated as described above. Calculations were based on four wild-type and four Fgf10 mutant embryos, scoring four sections per embryo. A 70% reduction was observed in the proportion of proliferative PDX1-positive cells within the pancreatic epithelium of Fgf10−/− embryos as compared to wild-type littermates (Student's t-test, P<0.0001). Thus while neither an increase in apoptosis nor any accelerated differentiation of pancreatic cells was observed, a significant decrease in pancreatic progenitor cell proliferation was detected, labelled by PDX1. This indicates that the reduction in the pancreatic epithelium observed in the Fgf10 mutant embryos is likely to be a result of underproliferation of pancreatic epithelial cells.

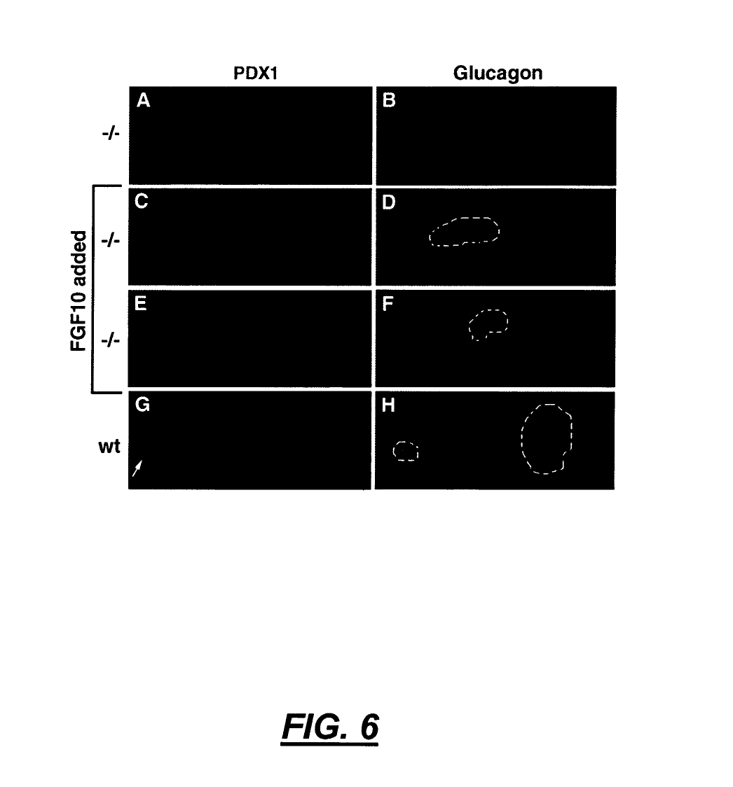

These results indicate that FGF10-driven proliferation is required to generate a quantitatively normal pool of epithelial progenitor cells, and in the absence of Fgf10, PDX1-positive progenitor cells are lost. It was investigated whether exogenously supplied FGF10 could rescue this pool of progenitor epithelial cells in Fgf10 mutant embryos. The gastrointestinal tracts from E9.5 wild-type littermates and Fgf10 mutant embryos were isolated and cultured in collagen gel with or without addition of recombinant FGF10 to the culture medium. After2 days, the explanted cultured tissue were fixed, embedded in paraffin wax and processed for immunohistology for PDX1 and glucagon. No PDX1-positive cells were detected in cultured explants derived from Fgf10 mutant embryos, although glucagon-positive cells were readily observed (FIGS. 6A,B). However, a significant number of PDX1-positive cells was observed in explants derived from Fgf10 mutant embryos cultured in the presence of FGF10. These PDX1-labelled cells formed an epithelial bud on one side of the duodenum (FIG. 6C); an adjacent section showed that glucagon-positive cells were present in the mesenchyme adjacent to the bud containing the PDX1-positive cells (FIG. 6D). An additional set of PDX1-positive cells was detected on the opposite side of the duodenum in these explanted tissue (FIG. 6E); however, no glucagon-positive cells were detected adjacent to these PDX1-positive cells (FIG. 6F). Cultured explants derived from wild-type embryos displayed similar PDX1-positive epithelial buds (FIGS. 6G,H). Since differentiation of glucagon-expressing cells normally occurs in the dorsal bud approximately 2 days before it does in the ventral bud, the two populations of PDX1-positive cells observed in vitro could represent the two different buds. These results demonstrate that in organ cultures, soluble FGF10 is capable of rescuing the epithelial progenitor cells in the Fgf10 mutant embryos.

The foregoing is illustrative of the present invention, and is not to be construed as limiting thereof. The invention is defined by the following claims, with equivalents of the claims to be included therein.

That which is claimed is:

1. A method of facilitating the proliferation of pancreatic progenitor cells, comprising administering a FGF polypeptide to said cells in an amount effective to facilitate the proliferation thereof.

2. The method according to claim 1, wherein said administering step is carried out in vitro.

3. The method according to claim 1, wherein said pancreatic progenitor cells are mammalian pancreatic progenitor cells.

4. The method according to claim 1, wherein said pancreatic progenitor cells are human pancreatic progenitor cells.

5. The method according to claim 1, wherein said FGF polypeptide is selected from the group consisting of FGF-2, FGF-7, and FGF-10 polypeptides.

6. The method according to claim 1, wherein said FGF polypeptide is an FGF-10 polypeptide.

7. A method of facilitating the proliferation of mammalian pancreatic progenitor cells in vitro, comprising administering an FGF polypeptide to said cells in vitro in an amount effective to facilitate the proliferation thereof.

8. The method according to claim 7, wherein said pancreatic progenitor cells are human pancreatic progenitor cells.

9. The method according to claim 7, wherein said FGF polypeptide is selected from the group consisting of FGF-2, FGF-7, and FGF-10 polypeptides.

10. The method according to claim 7, wherein said FGF polypeptide is an FGF-10 polypeptide.

11. The method according to claim 7, wherein said FGF polypeptide is administered in an amount ranging from about 0.5 ng/ml to about 1000 ng/ml.

12. The method according to claim 7, wherein said FGF polypeptide is administered in an amount ranging from about 1 ng/ml to about 500 ng/ml.

13. Isolated mammalian pancreatic progenitor cells produced by the process of claim 7.
